(19) United States
(12) Patent Application Publication
(10) Pub. No.: US 2026/0175189 A1
(43) Pub. Date: Jun. 25, 2026

(54) FULLY-CONTINUOUS FLOW SYNTHESIS METHOD OF GLYPHOSATE

(71) Applicant: Jiangxi Normal University, Nanchang (CN)

(72) Inventors: Fener CHEN, Shanghai (CN); Xianjing ZHENG, Shanghai (CN); Li WAN, Shanghai (CN); Minjie LIU, Shanghai (CN); Yajiao ZHANG, Shanghai (CN)

(21) Appl. No.: 19/539,139

(22) Filed: Feb. 13, 2026

(51) Int. Cl. B01J 19/00; B01J 19/12; B01J 19/24; C07D 487/04

(52) U.S. Cl. CPC B01J 19/0066; B01J 19/0053; B01J 19/0093; B01J 19/126; B01J 19/129; B01J 19/2415; B01J 19/2425; C07D 487/04; B01J 2219/00027; B01J 2219/00783; B01J 2219/00822; B01J 2219/00835; B01J 2219/0086; B01J 2219/00909

(57) ABSTRACT

A fully-continuous flow synthesis method of glyphosate is provided, which is performed by using a fully-continuous system including a plurality of pumps, a micromixer, a plurality of microchannel reactors, a plurality of tubular reactors, two back pressure valves, a plurality of reaction vessels, and a plurality of continuous crystallizers. Paraformaldehyde is depolymerized in the presence of an organic base, and the resultant depolymerization product undergoes an addition reaction with glycine to yield N, N-dihydroxymethylglycine, which further undergoes esterification with dimethyl phosphite (added in two batches) to generate a glyphosate ester. The glyphosate ester is adjusted to be acidic, hydrolyzed, desolvated, neutralized with a base, and purified to obtain glyphosate.

### TECHNICAL FIELD

This application relates to organic synthesis, and more particularly to a fully-continuous flow synthesis method of glyphosate.

### BACKGROUND

Glyphosate is an organophosphorus pesticide with the widest application range due to an outstanding weed control performance and a low price. Since its launch in the 1960s, glyphosate has quickly become the world's best-selling pesticide. In 2023, a global total production capacity of glyphosate was 1.18 million tons per year, with China's production capacity reaching 743,000 tons per year, accounting for 62.97% of the global glyphosate output. At present, the synthesis method of glyphosate mainly adopts a glycine method and an iminodiacetic acid (IDA) method. The glycine method is the main production method used in China. Glycine, polyformaldehyde and dimethyl phosphite are used as raw materials for reaction in the presence of triethylamine to obtain glyphosate. However, this method has always resulted in a low yield (generally 75-85%) and a low purity due to numerous by-products. The production process always adopts reaction vessels, which struggles with drawbacks of a serious three-waste pollution, a long reaction cycle, a high energy consumption and an unsafe essence. These drawbacks seriously affect quality of glyphosate per batch, resulting in uneven product quality, and is thus not conducive to industrial production.

Currently, the industrial production of glyphosate completely depends on the batch synthesis, failing to achieving the continuous preparation. It has never been reported about the continuous synthesis of methyl glyphosate from paraformaldehyde, and thus the drawbacks of the batch synthesis cannot be completely eliminated, resulting in poor inter-batch product stability and quality, low yield and high cost.

### SUMMARY

An object of the present disclosure is to provide a fully-continuous flow synthesis method of glyphosate with high production efficiency, and excellent product quality and consistency, so as to overcome the problems of uneven product quality, low purity caused by numerous by-products and complicated production process in the existing reaction-vessel-type production processes for glyphosate.

A fully-continuous flow synthesis method of glyphosate using a fully-continuous flow synthesis system, the fully-continuous flow synthesis system comprising a first pump, a second pump, a micromixer, a first microchannel reactor, a second microchannel reactor, a third microchannel reactor, a fourth microchannel reactor, a first tubular reactor, a second tubular reactor, a third tubular reactor, a first back pressure valve, a second back pressure valve, a first reaction vessel, a second reaction vessel, a third reaction vessel, a fourth reaction vessel, a fifth reaction vessel, a sixth reaction vessel, a first continuous crystallizer, a second continuous crystallizer, and a third continuous crystallizer; and the fully-continuous synthesis method comprising:
(1) dissolving paraformaldehyde in a first solvent to obtain a first reactant liquid; mixing the first reactant liquid with a second reactant liquid in the micromixer, followed by depolymerization in the first tubular reactor to obtain a depolymerization product, wherein the second reactant liquid is a first base or a solution of the first base in a second solvent; feeding the depolymerization product to the first reaction vessel; feeding, by a dual-screw feeder with a weighing function, glycine solid under a positive pressure created by a nitrogen, so as to prevent the glycine solid from coming into contact with air and absorbing moisture to form lumps during a long-term operation of the dual-screw feeder, thereby affecting a feeding accuracy, to the first reaction vessel followed by addition reaction under stirring to generate a first reaction mixture which contains N, N-dihydroxymethylglycine, wherein a sedimentation rate of the glycine solid is adjusted by controlling a stirring rate of the first reaction vessel to be identical to a downward flow rate of the depolymerization product; transporting the first reaction mixture from an outlet at a bottom of the first reaction vessel to a bottom of the second reaction vessel under the action of gravity, wherein the bottom of the first reaction vessel is located above the bottom of the second reaction vessel;
(2) quantitatively transferring, by a first pump, the N, N-dihydroxymethylglycine-containing reaction mixture from a top of the second reaction vessel to the first microchannel reactor; transporting, by a second pump, a part of phosphite ester to the first microchannel reactor; mixing the N, N-dihydroxymethylglycine-containing reaction mixture with the phosphite ester in the first microchannel reactor followed by esterification in the second microchannel reactor and the third microchannel reactor, so as to obtain a preliminarily-esterified product, wherein a second base is introduced into the second microchannel reactor, and the second base is the same as the first base; and another part of the phosphite ester is introduced into the third microchannel reactor; transporting the preliminarily-esterified product to the second tubular reactor for continuous esterification reaction to ensure complete conversion, so as to obtain a completely-esterified product; and
(3) transporting the completely-esterified product to pass through the first back pressure valve to enter the fourth microchannel reactor followed by cooling; introducing an acid from a middle of the fourth microchannel reactor followed by neutralization reaction to obtain a second reaction mixture, wherein the cooling is performed to remove a reaction heat generated by the neutralization reaction and avoid additional by-products caused by temperature fluctuations; transporting the second reaction mixture to the third tubular reactor, and heating the second reaction mixture to a preset temperature for hydrolysis reaction to obtain a third reaction mixture; transporting the third reaction mixture to the third reaction vessel through the second back pressure valve for desolventization to remove solvents and low-boiling-point compounds, so as to obtain a desolvated product; transporting the desolvated product sequentially to the fourth reaction vessel and the fifth reaction vessel for complete hydrolysis to obtain a fourth reaction mixture, wherein temperatures of the fourth reaction vessel and the fifth reaction vessel are elevated to continuously remove water and promote hydrolysis; transporting the fourth reaction mixture to the third sixth reaction followed by pH adjustment with a third base in a liquid state and sequential transportation to a first continuous crystallizer, a second continuous crystallizer and a third continuous crystallizer for gradient cooling crystallization to a crude glyphosate product; and subjecting the crude glyphosate product to filtration and drying to produce a glyphosate finished product with a purity greater than 99% and a total yield greater than 88% calculated based on glycine.

In some embodiments, the first base, the second base and the third base are each independently selected from the group consisting of liquid ammonia, triethylamine, trimethylamine, tributylamine, sodium methoxide, sodium ethoxide and potassium tert-butoxide;
in step (2), the phosphite ester is selected from the group consisting of dimethyl phosphorite, diethyl phosphorite, dipropyl phosphorite and dibutyl phosphorite; and
in step (3), the acid is selected from the group consisting of a 10-37 wt. % aqueous hydrogen chloride solution, a 10-40 wt. % aqueous phosphoric acid solution, formic acid and acetic acid.

In some embodiments, in step (1), the first solvent and the second solvent are each independently selected from the group consisting of pentanol, n-butanol, isobutanol, tert-butanol, n-propanol, isopropanol, ethanol, methanol, acetone and methyl isobutyl ketone.

In some embodiments, in step (2), the phosphite ester is fed into the first microchannel reactor in a solvent-free manner or in the presence of a third solvent;
in step (3), the acid is fed into the fourth microchannel reactor in a solvent-free manner or in the presence of a fourth solvent; and
the second solvent, the third solvent and the fourth solvent are each independently selected from the group consisting of water, n-propanol, isopropanol, ethanol, methanol and methyl isobutyl ketone.

In some embodiments, in step (1), a molar ratio of the first base to the glycine solid is 0.00001-1.0:1, and a molar ratio of the paraformaldehyde to the glycine solid is 1.0-3.0:1; and
in step (2), a molar ratio of the phosphite ester to the glycine solid is 1.0-1.4:1, a molar ratio of the second base to the glycine solid is 0.1-0.35:1, and a molar ratio of the acid to the glycine solid is 1.0-10:1.

In some embodiments, the depolymerization is performed in the first tubular reactor at 25-60° C. for 5-15 min;
the continuous esterification reaction in the second tubular reactor is performed at 60-85° C. for 10-20 min;
the hydrolysis reaction in the third tubular reactor is performed at 100-125° C. for 5-30 s;
the esterification reaction in the first microchannel reactor is performed at 55-70° C. for 3-8 min;
the esterification reaction in the second microchannel reactor is performed at 55-75° C. for 2-5 min;
the esterification reaction in the third microchannel reactor is performed at 60-75° C. for 1-5 min;
the neutralization reaction in the fourth microchannel reactor is performed at 0-10° C. for 1-5 min;
the addition reaction in the first reaction vessel is performed at 40-55° C. for 5-10 min;
a temperature of the second reaction vessel is controlled at 40-55° C., and a residence time of the first reaction mixture in the second reaction vessel is 5-10 min;
the desolventization in the third reaction vessel is performed at 80-150° C. for 10-40 min;
the hydrolysis reaction in the fourth reaction vessel is performed at 80-150° C. for 10-40 min;
the hydrolysis reaction in the fifth reaction vessel is performed at 90-150° C. for 10-40 min;
the pH adjustment in the sixth reaction vessel is performed at 55-75° C. for 5-10 min;
the first continuous crystallizer is controlled at 45-75° C., and a residence time in the first continuous crystallizer is 30-60 min;
the second continuous crystallizer is controlled at 35-60° C., and a residence time in the second continuous crystallizer is 30-60 min; and
the third continuous crystallizer is controlled at 20-45° C., and a residence time in the third continuous crystallizer is 30-60 min.

In some embodiments, in step (1), the micromixer is a high-shear online intensification reactor with a rotating rate of 800-3,000 rpm and a residence time of 3-60 s;
the dual-screw feeder is provided with a funnel, wherein the funnel is provided with an auxiliary stirrer to prevent solid materials from lumping and bridging, affecting the accuracy and uniformity of the transportation; the funnel has an online weighing and automatic calibration control mechanism with a control error of no more than 0.5%; the funnel is provided with a cover configured to seal the funnel; an upper portion of the cover is connected to a nitrogen inlet to introduce nitrogen into the dual-screw feeder to maintain the positive pressure, avoiding back-mixing and preventing the solid materials from coming into contact with air and absorbing moisture to form lumps during a long-term operation of the dual-screw feeder, affecting a feeding accuracy;
the first reaction vessel has an aspect ratio of 6-20:1, and is provided with a stirring shaft with 4-8 blades;
the second reaction vessel has an aspect ratio of 4-12:1, and is provided with a stirring shaft with 3-6 blades;
a liquid holdup of the first reaction vessel is 1-5 times that of the second reaction vessel; and
a median line height of the first reaction vessel is 20% or more higher than a median line height of the second reaction vessel;
in step (3), the third reaction vessel, the fourth reaction vessel, the first continuous crystallizer, the second continuous crystallizer and the third continuous crystallizer each have an aspect ratio of 4-12:1, an inlet size of 1.0-50 mm, an outlet size of 1.0-50 mm, and a height of 5.0-100 mm; each of the third reaction vessel, the fourth reaction vessel, the first continuous crystallizer, the second continuous crystallizer and the third continuous crystallizer is provided with a mixing chamber having a diameter of 5.0-500 mm and a stirring shaft with 3-6 blades; and
the first microchannel reactor, the second microchannel reactor and the third microchannel reactor each have a plate or tubular microchannel structure with an inner diameter of 1.0-100 mm and a length of 10-10,000 m.

In some embodiments, each of the first tubular reactor, the second tubular reactor and the third tubular reactor has an inner diameter of 10-200 mm, and is provided with a static mixing component;
each of the first microchannel reactor, the second microchannel reactor and the third microchannel reactor has a characteristic dimension less than 1 mm, and adopts a split-and-recombine configuration with a maximum inner diameter or a maximum side length less than 6 mm;
each of the third reaction vessel, the fourth reaction vessel and the fifth reaction vessel has a diameter of 5-1,000 mm and an aspect ratio of 5-50:1; and
each of the first continuous crystallizer, the second continuous crystallizer and the third continuous crystallizer has a diameter of 10-1,000 mm and an aspect ratio of 10-50:1.

In some embodiments, a pressure of the first back pressure valve is 1-10 bar, and a pressure of the second back pressure valve is 1-20 bar.

In some embodiments, in step (3), the gradient cooling crystallization is performed in the presence of a solvent selected from the group consisting of water, methanol, ethanol, n-propanol and isopropanol.

Compared to the prior art, the present disclosure has the following beneficial effects.

(1) The first, second and third reaction vessels with a high aspect ratio achieve a high mass transfer efficiency. Local back-mixing enables rapid mixing of materials while maintaining good compatibility with a solid-containing system. Additionally, the high aspect ratio effectively shortens the heat transfer distance from a center of the reaction vessel to a wall of the reaction vessel, significantly improving the heat transfer efficiency and preventing noticeable temperature differences within the reaction vessel.

(2) The present disclosure realizes fully-continuous flow operation from the feeding of raw materials and reagents to the reaction engineering and crystallization process, greatly reducing manual intervention, enabling automated continuous production and ensuring the stability of product quality and yield.

(3) The present disclosure provides a glyphosate finished product with a purity greater than 99% and a total yield greater than 88% calculated based on glycine, demonstrating significant improvements compared to traditional reaction-vessel-type processes.

### BRIEF DESCRIPTION OF THE DRAWINGS

FIG. 1 is a flow chart of a fully-continuous flow synthesis method of glyphosate according to an embodiment of the present disclosure; and

FIG. 2 is a schematic diagram of a solid feeding control system according to an embodiment of the present disclosure.

### DETAILED DESCRIPTION OF EMBODIMENTS

The present disclosure will be further described with reference to the accompanying drawings and embodiments. The embodiments disclosed herein are merely illustrative of the disclosure, and are not intended to limit the present disclosure.

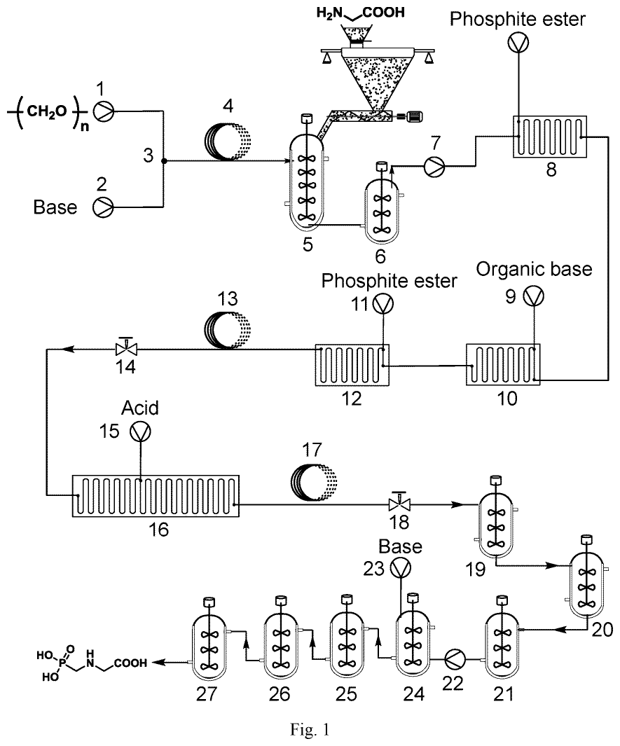

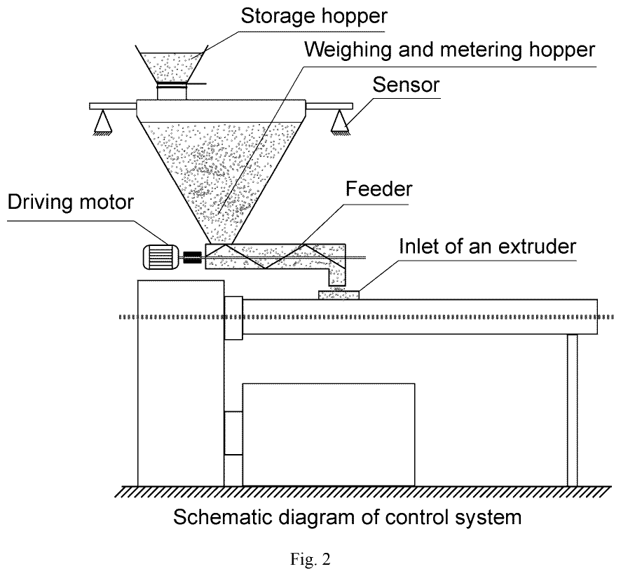

This application provides a fully-continuous flow synthesis of glyphosate and a solid feeding control system, which are schematically shown in FIG. 1 and FIG. 2, respectively.

### Example 1

Paraformaldehyde (0.14 kg/L, 2.0 eq) was dissolved in methanol to obtain a first reactant liquid. Sodium methoxide (0.0001 eq) was dissolved in methanol to obtain a second reactant liquid. The first reactant liquid and the second reactant liquid were transported into a micromixer 3 through a first pump 1 and a second pump 2, respectively. The first reactant liquid was fully mixed with the second reactant liquid in the micromixer 3 (a high-shear online intensification reactor) at a rotating rate of 1,000 rpm for a residence time of 15 s, followed by depolymerization in a first tubular reactor 4 at 50° C. for 5 min to obtain a depolymerization product. The depolymerization product was fed to a first reaction vessel 5. Glycine solid (0.8 kg/L, 1.0 eq) was accurately fed, by a dual-screw feeder with a weighing function under a positive pressure created by a nitrogen atmosphere to the first reaction vessel 5, so as to prevent the glycine solid from coming into contact with air and absorbing moisture to form lumps during a long-term operation of the dual-screw feeder, thereby affecting a feeding accuracy. Subsequently, an addition reaction was performed at 50° C. for 8 min in the first reaction vessel 5 with a high aspect ratio to generate a first reaction mixture which contained relatively less N, N-dihydroxymethylglycine. The first reaction mixture was transported from an outlet at a bottom of the first reaction vessel 5 to a bottom of a second reaction vessel 6 under the action of gravity, followed by continuous addition reaction at 55° C. for 7 min to generate a second reaction mixture which contained more N, N-dihydroxymethylglycine.

The N, N-dihydroxymethylglycine-containing reaction mixture flowing out of the second reaction vessel 6 was quantitatively transferred to a first microchannel reactor 8 by a third pump 7, and a part of dimethyl phosphite (0.3 kg/L, 1.4 eq) was transported to the first microchannel reactor 8, followed by esterification at 55° C. for 4 min to generate a third reaction mixture which contained relatively less methyl glyphosate. The third reaction mixture was transported to a second microchannel reactor 10, and mixed with a first base transferred by a fourth pump 9 followed by esterification at 65° C. for 5 min to generate a fourth reaction mixture which contained more methyl glyphosate. The fourth reaction mixture was transported to a third microchannel reactor 12. Another part of dimethyl phosphite was introduced into the third microchannel reactor 12 by a fifth pump 11, and fully mixed with the fourth reaction mixture followed by esterification at 70° C. for 5 min to obtain a fifth reaction mixture which contained more and more methyl glyphosate. The fifth reaction mixture was transported to a second tubular reactor 13 followed by continuous esterification reaction at 80° C. for 12 min to ensure complete conversion of materials, so as to completely obtain methyl glyphosate. The methyl glyphosate-containing reaction mixture flowed out of the second tubular reactor 13, and entered a first back pressure valve 14 (3 bar), where the first back pressure valve 14 was configured to respectively control reaction pressures in the first microchannel reactor 8, the second microchannel reactor 10, the third microchannel reactor 12 and the second tubular reactor 13.

20 wt. % hydrogen chloride solution was introduced into a front portion of a fourth microchannel reactor 16 by a sixth pump 15 for precooling, and fully mixed with the methyl glyphosate-containing reaction mixture in a rear portion of the fourth microchannel reactor 16 followed by neutralization reaction at 0° C. for 3 min to obtain a sixth reaction mixture. The sixth reaction mixture was transported to a third tubular reactor 17 for rapidly heating to 100° C., then entering a second back pressure valve 18 (5 bar), where the second back pressure valve 18 was configured to respectively control reaction pressures in the fourth microchannel reactor 16 and the third tubular reactor 17. The sixth reaction mixture was transported to a third reaction vessel 19 followed by desolventization at 110° C. for 28 min to remove solvents and low-boiling-point methanol as well as by-product chloromethane, so as to obtain a desolvated product. The desolvated product was transported to a fourth reaction vessel 20 for preliminary hydrolysis at 115° C. for 35 min, and continuously transported to a fifth reaction vessel 21 for complete hydrolysis reaction at 125° C. for 35 min, so as to obtain a seventh reaction mixture. During the preliminary and complete hydrolysis, temperatures of the fourth reaction vessel 20 and the fifth reaction vessel 21 are elevated to continuously remove water and promote hydrolysis. The seventh reaction mixture was transported to a sixth reaction vessel 24 by a seventh pump 22, and fully mixed with a second base in a liquid state transferred by an eighth pump 23 for adjusting pH to 2.0, followed by sequential transportation to a first continuous crystallizer 25 (50° C.), a second continuous crystallizer 26 (35° C.) and a third continuous crystallizer 27 (25° C.) for gradient cooling crystallization to obtain a crude glyphosate product. The crude glyphosate product was subjected to filtration and drying to produce a glyphosate finished product with a purity of 97%, a total yield of 88% calculated based on glycine. The total reaction time was 105.5 min.

### Example 2

Paraformaldehyde (0.15 kg/L, 2.0 eq) was dissolved in methanol to obtain a first reactant liquid. Sodium ethoxide (0.001 eq) was dissolved in methanol to obtain a second reactant liquid. The first reactant liquid and the second reactant liquid were transported into the micromixer 3 by the first pump 1 and the second pump 2, respectively. The first reactant liquid was fully mixed with the second reactant liquid in the micromixer 3 (a high-shear online intensification reactor) at a rotating rate of 1,500 rpm for a residence time of 10 s, followed by depolymerization in the first tubular reactor 4 at 50° C. for 5 min to obtain a depolymerization product. The depolymerization product was fed to the first reaction vessel 5. Glycine solid (0.85 kg/L, 1.0 eq) was accurately fed, by the dual-screw feeder with the weighing function under the positive pressure created by the nitrogen atmosphere to the first reaction vessel 5 with a high aspect ratio, so as to prevent the glycine solid from coming into contact with air and absorbing moisture to form lumps during the long-term operation of the dual-screw feeder, thereby affecting a feeding accuracy. Subsequently, triethylamine (0.8 eq) was added to the first reaction vessel 5, followed by addition reaction at 55° C. for 8 min to generate the first reaction mixture which contained relatively less N, N-dihydroxymethylglycine. The first reaction mixture was transported from an outlet at the bottom of the first reaction vessel 5 to the bottom of the second reaction vessel 6 under the action of gravity, followed by continuous addition reaction at 55° C. for 6.8 min to generated a second reaction mixture which contained more N, N-dihydroxymethylglycine.

The N, N-dihydroxymethylglycine-containing reaction mixture flowing out of the second reaction vessel 6 was quantitatively transferred to the first microchannel reactor 8 by the third pump 7, and a part of dimethyl phosphite (0.3 kg/L, 1.2 eq) was transported to the first microchannel reactor 8, followed by esterification at 60° C. for 4 min to generate a third reaction mixture which contained relatively less methyl glyphosate. The third reaction mixture was transported to the second microchannel reactor 10, and mixed with a first base transferred by the fourth pump 9, followed by esterification at 65° C. for 4 min to generate a fourth reaction mixture which contained more methyl glyphosate. The fourth reaction mixture was transported to the third microchannel reactor 12. Another part of dimethyl phosphite was introduced into the third microchannel reactor 12 by the fifth pump 11, and fully mixed with the fourth reaction mixture followed by esterification at 70° C. for 3 min to obtain a fifth reaction mixture which contained more and more methyl glyphosate. The fifth reaction mixture was transported to the second tubular reactor 13 followed by continuous esterification reaction at 75° C. for 15 min, so as to ensure complete conversion of materials, so as to completely obtain methyl glyphosate. The methyl glyphosate-containing reaction mixture flowed out of the second tubular reactor 13, and entered the first back pressure valve 14 (5 bar), where the first back pressure valve 14 was configured to respectively control reaction pressures in the first microchannel reactor 8, the second microchannel reactor 10, the third microchannel reactor 12 and the second tubular reactor 13.

30 wt. % hydrogen chloride solution was introduced into the front portion of the fourth microchannel reactor 16 by the sixth pump 15 for precooling, and fully mixed with the methyl glyphosate-containing reaction mixture in the rear portion of the fourth microchannel reactor 16 followed by neutralization reaction at 5° C. for 4 min to obtain a sixth reaction mixture. The sixth reaction mixture was transported to the third tubular reactor 17 for rapidly heating to 110° C., then entering the second back pressure valve 18 (6 bar), where the second back pressure valve 18 was configured to respectively control reaction pressures in the fourth microchannel reactor 16 and the third tubular reactor 17. The sixth reaction mixture was transported to the third reaction vessel 19 followed by desolventization at 110° C. for 30 min to remove solvents and low-boiling-point methanol as well as by-product chloromethane to obtain a desolvated product. The desolvated product was transported to the fourth reaction vessel 20 for preliminary hydrolysis at 118° C. for 40 min, and continuously transported to the fifth reaction vessel 21 for complete hydrolysis reaction at 120° C. for 40 min, so as to obtain a seventh reaction mixture. During the preliminary and complete hydrolysis, temperatures of the fourth reaction vessel 20 and the fifth reaction vessel 21 are elevated to continuously remove water and promote hydrolysis. The seventh reaction mixture was transported to the sixth reaction vessel 24 by the seventh pump 22, and fully mixed with a second base in a liquid state transferred by the eighth pump 23 for adjusting pH to 2.2, followed by sequential transportation to the first continuous crystallizer 25 (55° C.), the second continuous crystallizer 26 (30° C.) and the third continuous crystallizer 27 (25° C.) for gradient cooling crystallization to obtain a crude glyphosate product. The crude glyphosate product was subjected to filtration and drying to produce a glyphosate finished product with a purity of 98%, a total yield of 87% calculated based on glycine. The total reaction time was 105.5 min.

### Example 3

Paraformaldehyde (0.15 kg/L, 2.0 eq) was dissolved in methanol to obtain a first reactant liquid. Triethylamine (0.8 eq) was dissolved in methanol to obtain a second reactant liquid. The first reactant liquid and the second reactant liquid were transported into the micromixer 3 through the first pump 1 and the second pump 2, respectively. The first reactant liquid was fully mixed with the second reactant liquid in the micromixer 3 (a high-shear online intensification reactor) at a rotating rate of 1,800 rpm for a residence time of 9 s, followed by depolymerization in the first tubular reactor 4 at 50° C. for 4 min to obtain a depolymerization product. The depolymerization product was fed to the first reaction vessel 5. Glycine solid (0.8 kg/L, 1.0 eq) was accurately fed, by the dual-screw feeder with the weighing function under the positive pressure created by the nitrogen atmosphere to the first reaction vessel 5 with a high aspect ratio, so as to prevent the glycine solid from coming into contact with air and absorbing moisture to form lumps during a long-term operation of the dual-screw feeder, thereby affecting a feeding accuracy. Subsequently, an addition reaction was performed at 52° C. for 8.2 min to generate a first reaction mixture which contained relatively less N, N-dihydroxymethylglycine. The first reaction mixture was transported from an outlet at the bottom of the first reaction vessel 5 to the bottom of the second reaction vessel 6 under the action of gravity, followed by addition reaction at 55° C. for 6.8 min to generate a second reaction mixture which contained more N, N-dihydroxymethylglycine.

The N, N-dihydroxymethylglycine-containing reaction mixture flowing out of the second reaction vessel 6 was quantitatively transferred to the first microchannel reactor 8 by the third pump 7, and a part of dimethyl phosphite (0.32 kg/L, 1.2 eq) was transported to the first microchannel reactor 8, followed by esterification at 65° C. for 3 min to generate a third reaction mixture which contained relatively less methyl glyphosate. The third reaction mixture was transported to the second microchannel reactor 10, and mixed with a first base transferred by the fourth pump 9 followed by esterification at 65° C. for 3.5 min to generate a fourth reaction mixture which contained more methyl glyphosate. Another part of dimethyl phosphite was introduced to the third microchannel reactor 12 by the fifth pump 11, and fully mixed with the fourth reaction mixture in the third microchannel reactor 12 followed by esterification reaction at 75° C. for 3 min to obtain a fifth reaction mixture which contained more and more methyl glyphosate. The fifth reaction mixture was transported to the second tubular reactor 13 followed by continuous esterification reaction at 80° C. for 12 min to ensure complete conversion of materials, so as to completely obtain methyl glyphosate. The methyl glyphosate-containing reaction mixture flowed out of the second tubular reactor 13, and entered the first back pressure valve 14 (7 bar), where the first back pressure valve 14 was configured to respectively control reaction pressures in the first microchannel reactor 8, the second microchannel reactor 10, the third microchannel reactor 12 and the second tubular reactor 13.

37 wt. % hydrogen chloride solution was introduced into the front portion of the fourth microchannel reactor 16 by the sixth pump 15 for precooling, and fully mixed with the methyl glyphosate-containing reaction mixture in the rear portion of the fourth microchannel reactor 16 followed by neutralization reaction at 10° C. for 2 min to obtain a sixth reaction mixture. The sixth reaction mixture was transported to the third tubular reactor 17 for rapidly heating to 120° C., then entering the second back pressure valve 18 (4 bar), where the second back pressure valve 18 was configured to respectively control reaction pressures in the fourth microchannel reactor 16 and the third tubular reactor 17. The sixth reaction mixture was transported to the third reaction vessel 19 followed by desolventization at 110° C. for 30 min to remove solvents and low-boiling-point methanol as well as by-product chloromethane, so as to obtain a desolvated product. The desolvated product was transported to the fourth reaction vessel 20 for preliminary hydrolysis at 118° C. for 35 min, and continuously transported to the fifth reaction vessel 21 for complete hydrolysis reaction at 120° C. for 38 min, so as to obtain a seventh reaction mixture. During the preliminary and complete hydrolysis, temperatures of the fourth reaction vessel 20 and the fifth reaction vessel 21 are elevated to continuously remove water and promote hydrolysis. The seventh reaction mixture was transported to the sixth reaction vessel 24 by the seventh pump 22, and fully mixed with a second base in a liquid state transferred by the eighth pump 23 for adjusting pH to 1.8, followed by sequential transportation to the first continuous crystallizer 25 (55° C.), the second continuous crystallizer 26 (35° C.) and the third continuous crystallizer 27 (25° C.) for gradient cooling crystallization to obtain a crude glyphosate product. The crude glyphosate product was subjected to filtration and drying to obtain a glyphosate finished product with a purity of 99%, a total yield of 88% calculated based on glycine. The total reaction time was 115.5 min.

### Example 4

Paraformaldehyde (0.12 kg/L, 2.0 eq) was dissolved in methanol to obtain a first reactant liquid. Tributylamine (0.8 eq) was dissolved in methanol to obtain a second reactant liquid. The first reactant liquid and the second reactant liquid were transported into the micromixer 3 through the first pump 1 and the second pump 2, respectively. The first reactant liquid was fully mixed with the second reactant liquid in the micromixer 3 (a high-shear online intensification reactor) at a rotating rate of 2,000 rpm for a residence time of 8 s, followed by depolymerization in the first tubular reactor 4 at 55° C. for 3.5 min to obtain a depolymerization product. The depolymerization product was fed to the first reaction vessel 5. Glycine solid (0.65 kg/L, 1.0 eq) was accurately fed, by the dual-screw feeder with the weighing function under the positive pressure created by the nitrogen atmosphere to the first reaction vessel 5, so as to prevent the glycine solid from coming into contact with air and absorbing moisture to form lumps during the long-term operation of the dual-screw feeder, thereby affecting a feeding accuracy. Subsequently, an addition reaction was performed at 50° C. for 8 min in the first reaction vessel 5 with a high aspect ratio to generate a first reaction mixture which contained less N, N-dihydroxymethylglycine. The first reaction mixture was transported from an outlet at the bottom of the first reaction vessel 5 to the bottom of the second reaction vessel 6 under the action of gravity, followed by continuous addition reaction at 55° C. for 7 min to generate a second reaction mixture which contained more N, N-dihydroxymethylglycine.

The N, N-dihydroxymethylglycine-containing reaction mixture flowing out of the second reaction vessel 6 was quantitatively transferred to the first microchannel reactor 8 by the third pump 7, and a part of dimethyl phosphite (0.28 kg/L, 1.2 eq) was transported to the first microchannel reactor 8, followed by esterification reaction at 65° C. for 3.5 min to obtain a third reaction mixture which contained less methyl glyphosate. The third reaction mixture was transported to the second microchannel reactor 10, and mixed with a first base transferred by the fourth pump 9 followed by esterification reaction at 68° C. for 4 min to generate a fourth reaction mixture which contained more methyl glyphosate. The fourth reaction mixture was transported to the third microchannel reactor 12. Another part of dimethyl phosphite was introduced into the third microchannel reactor 12 by the fifth pump 11, and fully mixed with the fourth reaction mixture followed by esterification reaction at 75° C. for 2.8 min to obtain a fifth reaction mixture which contained more and more methyl glyphosate. The fifth reaction mixture was transported to the second tubular reactor 13 followed by continuous esterification reaction at 75° C. for 15 min to ensure complete conversion of materials, so as to completely obtain methyl glyphosate. The methyl glyphosate-containing reaction mixture flowed out of the second tubular reactor 13, and entering the first back pressure valve 14 (5 bar), where the first back pressure valve 14 was configured to respectively control reaction pressures in the first microchannel reactor 8, the second microchannel reactor 10, the third microchannel reactor 12 and the second tubular reactor 13.

31 wt. % hydrogen chloride solution was introduced into the front portion of the fourth microchannel reactor 16 by the sixth pump 15 for precooling, and fully mixed with the methyl glyphosate-containing reaction mixture in the rear portion of the fourth microchannel reactor 16 followed by neutralization reaction at 10° C. for 2 min to obtain a sixth reaction mixture. The sixth reaction mixture was transported to the third tubular reactor 17 for rapidly heating to 120° C., entering the second back pressure valve 18 (6 bar), where the second back pressure valve 18 was configured to respectively control reaction pressures in the fourth microchannel reactor 16 and the third tubular reactor 17. The sixth reaction mixture was transported to the third reaction vessel 19 followed by desolventization at 110° C. for 35 min to remove solvents and low-boiling-point methanol as well as by-product chloromethane, so as to obtain a desolvated product. The desolvated product was transported to the fourth reaction vessel 20 for preliminary hydrolysis reaction at 118° C. for 40 min, and continuously transported to the fifth reaction vessel 21 for complete hydrolysis reaction at 120° C. for 40 min, so as to obtain a seventh reaction mixture. During the preliminary and complete hydrolysis, temperatures of the fourth reaction vessel 20 and the fifth reaction vessel 21 are elevated to continuously remove water and promote hydrolysis. The seventh reaction mixture was transported to the sixth reaction vessel 24 by the seven pump 22, and fully mixed with a second base in a liquid state transferred by the eighth pump 23 for adjusting pH to 1.5, followed by sequential transportation to the first continuous crystallizer 25 (50° C.), the second continuous crystallizer 26 (30° C.) and the third continuous crystallizer 27 (25° C.) for gradient cooling crystallization to obtain a crude glyphosate product. The crude glyphosate product was subjected to filtration and drying to obtain a glyphosate finished product with a purity of 97%, a total yield of 86% based on glycine. The total reaction time was 115 min.

It should be noted that although the embodiments have been described in detail, the description should not be constructed as limitations to this present disclosure. Base on the creations of this application, any other modifications and changes made to the embodiments of the present disclosure, equivalent structures and equivalent variation made to the description and accompany figures of the present disclosure can be directly or indirectly applied to other arts, and shall fall within the scope of the present disclosure.

What is claimed is:

1. A fully-continuous flow synthesis method of glyphosate using a fully-continuous flow synthesis system, the fully-continuous flow synthesis system comprising a first pump, a second pump, a micromixer, a first microchannel reactor, a second microchannel reactor, a third microchannel reactor, a fourth microchannel reactor, a first tubular reactor, a second tubular reactor, a third tubular reactor, a first back pressure valve, a second back pressure valve, a first reaction vessel, a second reaction vessel, a third reaction vessel, a fourth reaction vessel, a fifth reaction vessel, a sixth reaction vessel, a first continuous crystallizer, a second continuous crystallizer, and a third continuous crystallizer; and the fully-continuous synthesis method comprising:
(1) dissolving paraformaldehyde in a first solvent to obtain a first reactant liquid; mixing the first reactant liquid with a second reactant liquid in the micromixer, followed by depolymerization in the first tubular reactor to obtain a depolymerization product, wherein the second reactant liquid is a first base or a solution of the first base in a second solvent; feeding the depolymerization product to the first reaction vessel; feeding, by a dual-screw feeder with a weighing function, glycine solid under a positive pressure created by a nitrogen atmosphere to the first reaction vessel followed by addition reaction under stirring to generate a first reaction mixture which contains N, N-dihydroxymethylglycine, wherein a sedimentation rate of the glycine solid is adjusted by controlling a stirring rate of the first reaction vessel to be identical to a downward flow rate of the depolymerization product; transporting the first reaction mixture from an outlet at a bottom of the first reaction vessel to a bottom of the second reaction vessel under the action of gravity, wherein the bottom of the first reaction vessel is located above the bottom of the second reaction vessel;
(2) quantitatively transferring, by a first pump, the N, N-dihydroxymethylglycine-containing reaction mixture from a top of the second reaction vessel to the first microchannel reactor; transporting, by a second pump, a part of a phosphite ester to the first microchannel reactor; mixing the N, N-dihydroxymethylglycine-containing reaction mixture with the phosphite ester in the first microchannel reactor followed by esterification in the second microchannel reactor and the third microchannel reactor, so as to obtain a preliminarily-esterified product, wherein a second base is introduced into the second microchannel reactor, and the second base is the same as the first base; and another part of the phosphite ester is introduced into the third microchannel reactor; transporting the preliminarily-esterified product to the second tubular reactor for continuous esterification reaction to ensure complete conversion, so as to obtain a completely-esterified product; and
(3) transporting the completely-esterified product to pass through the first back pressure valve to enter the fourth microchannel reactor followed by cooling; introducing an acid from a middle of the fourth microchannel reactor followed by neutralization reaction to obtain a second reaction mixture; transporting the second reaction mixture to the third tubular reactor, and heating the second reaction mixture to a preset temperature for hydrolysis reaction to obtain a third reaction mixture; transporting the third reaction mixture to the third reaction vessel through the second back pressure valve for desolventization to remove solvents, so as to obtain a desolvated product; transporting the desolvated product sequentially to the fourth reaction vessel and the fifth reaction vessel for complete hydrolysis to obtain a fourth reaction mixture, wherein temperatures of the fourth reaction vessel and the fifth reaction vessel are elevated to continuously remove water and promote hydrolysis; transporting the fourth reaction mixture to the sixth reaction vessel followed by pH adjustment with a third base in a liquid state and sequential transportation to a first continuous crystallizer, a second continuous crystallizer and a third continuous crystallizer for gradient cooling crystallization to a crude glyphosate product; and subjecting the crude glyphosate product to filtration and drying to produce a glyphosate finished product with a purity greater than 99% and a total yield greater than 88% calculated based on glycine.

2. The fully-continuous flow synthesis method of claim 1, wherein the first base, the second base and the third base are each independently selected from the group consisting of liquid ammonia, triethylamine, trimethylamine, tributylamine, sodium methoxide, sodium ethoxide and potassium tert-butoxide;
in step (2), the phosphite ester is selected from the group consisting of dimethyl phosphorite, diethyl phosphorite, dipropyl phosphorite and dibutyl phosphorite; and
in step (3), the acid is selected from the group consisting of a 10-37 wt. % aqueous hydrogen chloride solution, a 10-40 wt. % aqueous phosphoric acid solution, formic acid and acetic acid.

3. The fully-continuous flow synthesis method of claim 1, wherein in step (1), the first solvent and the second solvent are each independently selected from the group consisting of pentanol, n-butanol, isobutanol, tert-butanol, n-propanol, isopropanol, ethanol, methanol, acetone and methyl isobutyl ketone.

4. The fully-continuous flow synthesis method of claim 1, wherein in step (2), the phosphite ester is fed into the first microchannel reactor in a solvent-free manner or in the presence of a third solvent;
in step (3), the acid is fed into the fourth microchannel reactor in a solvent-free manner or in the presence of a fourth solvent; and
the second solvent, the third solvent and the fourth solvent are each independently selected from the group consisting of water, n-propanol, isopropanol, ethanol, methanol and methyl isobutyl ketone.

5. The fully-continuous flow synthesis method of claim 1, wherein in step (1), a molar ratio of the first base to the glycine solid is 0.00001-1.0:1, and a molar ratio of the paraformaldehyde to the glycine solid is 1.0-3.0:1; and
in step (2), a molar ratio of the phosphite ester to the glycine solid is 1.0-1.4:1, a molar ratio of the second base to the glycine solid is 0.1-0.35:1, and a molar ratio of the acid to the glycine solid is 1.0-10:1.

6. The fully-continuous flow synthesis method of claim 1, wherein the depolymerization is performed in the first tubular reactor at 25-60° C. for 5-15 min;
the continuous esterification reaction in the second tubular reactor is performed at 60-85° C. for 10-20 min;
the hydrolysis reaction in the third tubular reactor is performed at 100-125° C. for 5-30 s;
the esterification reaction in the first microchannel reactor is performed at 55-70° C. for 3-8 min;
the esterification reaction in the second microchannel reactor is performed at 55-75° C. for 2-5 min;
the esterification reaction in the third microchannel reactor is performed at 60-75° C. for 1-5 min;
the neutralization reaction in the fourth microchannel reactor is performed at 0-10° C. for 1-5 min;
the addition reaction in the first reaction vessel is performed at 40-55° C. for 5-10 min;
a temperature of the second reaction vessel is controlled at 40-55° C., and a residence time of the first reaction mixture in the second reaction vessel is 5-10 min;
the desolventization in the third reaction vessel is performed at 80-150° C. for 10-40 min;
the hydrolysis reaction in the fourth reaction vessel is performed at 80-150° C. for 10-40 min;
the hydrolysis reaction in the fifth reaction vessel is performed at 90-150° C. for 10-40 min;
the pH adjustment in the sixth reaction vessel is performed at 55-75° C. for 5-10 min;
the first continuous crystallizer is controlled at 45-75° C., and a residence time in the first continuous crystallizer is 30-60 min;
the second continuous crystallizer is controlled at 35-60° C., and a residence time in the second continuous crystallizer is 30-60 min; and
the third continuous crystallizer is controlled at 20-45° C., and a residence time in the third continuous crystallizer is 30-60 min.

7. The fully-continuous flow synthesis method of claim 1, wherein in step (1), the micromixer is an online intensification reactor with a rotating rate of 800-3,000 rpm and a residence time of 3-60 s;
the dual-screw feeder is provided with a funnel, wherein the funnel is provided with an auxiliary stirrer; the funnel has an online weighing and automatic calibration control mechanism with a control error of no more than 0.5%; the funnel is provided with a cover configured to seal the funnel; an upper portion of the cover is connected to a nitrogen inlet to introduce nitrogen into the dual-screw feeder to maintain the positive pressure;
the first reaction vessel has an aspect ratio of 6-20:1, and is provided with a stirring shaft with 4-8 blades;
the second reaction vessel has an aspect ratio of 4-12:1, and is provided with a stirring shaft with 3-6 blades;
a liquid holdup of the first reaction vessel is 1-5 times that of the second reaction vessel; and
a median line height of the first reaction vessel is 20% or more higher than a median line height of the second reaction vessel;
in step (3), the third reaction vessel, the fourth reaction vessel, the first continuous crystallizer, the second continuous crystallizer and the third continuous crystallizer each have an aspect ratio of 4-12:1, an inlet size of 1.0-50 mm, an outlet size of 1.0-50 mm, and a height of 5.0-100 mm; each of the third reaction vessel, the fourth reaction vessel, the first continuous crystallizer, the second continuous crystallizer and the third continuous crystallizer is provided with a mixing chamber having a diameter of 5.0-500 mm and a stirring shaft with 3-6 blades; and
the first microchannel reactor, the second microchannel reactor and the third microchannel reactor each have a plate or tubular microchannel structure with an inner diameter of 1.0-100 mm and a length of 10-10,000 m.

8. The fully-continuous flow synthesis method of claim 1, wherein each of the first tubular reactor, the second tubular reactor and the third tubular reactor has an inner diameter of 10-200 mm, and is provided with a static mixing component;
each of the first microchannel reactor, the second microchannel reactor and the third microchannel reactor has a characteristic dimension less than 1 mm, and adopts a split-and-recombine configuration with a maximum inner diameter or a maximum side length less than 6 mm;
each of the third reaction vessel, the fourth reaction vessel and the fifth reaction vessel has a diameter of 5-1,000 mm and an aspect ratio of 5-50:1; and
each of the first continuous crystallizer, the second continuous crystallizer and the third continuous crystallizer has a diameter of 10-1,000 mm and an aspect ratio of 10-50:1.

9. The fully-continuous flow synthesis method of claim 1, wherein a pressure of the first back pressure valve is 1-10 bar, and a pressure of the second back pressure valve is 1-20 bar.

10. The fully-continuous flow synthesis method of claim 1, wherein in step (3), the gradient cooling crystallization is performed in the presence of a solvent selected from the group consisting of water, methanol, ethanol, n-propanol and isopropanol.
